(19) United States
(12) Patent Application Publication
(10) Pub. No.: US 2006/0246522 A1
(43) Pub. Date: Nov. 2, 2006

(54) C-reactive protein immunoassay and method

(76) Inventors: Balwant S. Bhullar, La Vista, NE (US); Daniel T. Bourne, La Vista, NE (US)

Correspondence Address: MCHALE & SLAVIN, P.A., 2855 PGA BLVD, PALM BEACH GARDENS, FL, 33410, US

(21) Appl. No.: 11/118,756

(22) Filed: Apr. 28, 2005

(51) Int. Cl. G01N 33/53

(52) U.S. Cl. 435/7.92

(57) ABSTRACT

The present invention is directed towards the synthesis and/or use of a mixture of analyte capture reagents comprising antibodies and a non-immunological reagent provided on a solid phase assay, selected to screen for very high and low levels of an analyte. The non-immunological reagent comprising a PC-conjugate that contains multiple copies of covalently coupled phosphorylcholine (PC) moieties, particularly towards a phosphorylcholine-thyroglobulin conjugate assaying for C-reactive protein (CRP), a known inflammatory marker. The mixture of capture reagents function to eliminate hook effect (i.e. false negatives) when CRP is present in sample at high concentrations.

### FIELD OF THE INVENTION

The present invention is directed towards a conjugate complex for enhancing the density of phosphorylcholine (PC) sites to establish simultaneous multiple interactions with a C-Reactive Protein (CRP), particularly to the of binding CRP with a modified carrier protein forming a PC-conjugate, and most particularly to the incorporation of a mixture of PC-conjugate (non-immunological) and antibodies (immunological) reagents onto a one-step test strip to provide a wide range of sensitivity for the detection of CRP.

### BACKGROUND OF THE INVENTION

Preliminary C-Reactive Protein is an “acute-phase” protein in which concentrations in the serum, or plasma, increase rapidly from 3 mg/L in healthy adults to more than 300 mg/L in response to infectious or non-infectious inflammatory processes, i.e. rheumatoid arthritis, arteriosclerosis or other cardiovascular diseases. In clinical practice, CRP concentrations are routinely assayed to detect, monitor and/or predict a variety of inflammation-associated human disease. Current conventional nephelometeric and turbidmetric tests for CRP only allow the measurement of concentrations of 0.2 to 0.4 mg/dL with relative precision. Therefore, a need exists for a relatively simple and inexpensive testing device to rapidly identify apparently healthy persons at risk of developing disease.

There are numerous commercially available systems and/or devices for diagnostic testing of analytes in tissue samples. Lateral flow devices, or test strips, are known to provide simple and rapid semi-quantitative or quantitative detection. These test strips are part of a segment of in vitro diagnostics called Point of Care (POC) devices. The result obtained from test strips may be observed visually or quantitated with a simple detection apparatus such that a highly sophisticated apparatus is not required.

It is well known that sandwich immunoassays are prone to false negative results at high concentrations of analyte, as seen when testing diseased individuals with very high levels of CRP concentrations. This is attributable to the prozone or high-dose ‘hook effect’, which describes the inhibition of immune complex formation by excess analyte (antigen) concentrations that prevent cross-linking of antigen-antibody complexes, thus causing a decreasing signal with increasing analyte.

In the past, if the hook effect is suspected, appropriate dilutions of the sample are required to obtain an accurate estimation of CRP concentrations. Unfortunately, this approach is highly laborious, requiring trained technicians to perform the assay. Additionally, the incidence of dilution related errors increases with these techniques. Consequently, a need exists for a composition for use with conventional testing devices that will rapidly and accurately identify apparently healthy persons at risk of developing disease.

The CRP protein belongs to a family of cyclic pentameric proteins known as pentraxins. It is composed of five identical noncovalently bound subunits of 206 amino acids with a molecular mass of ˜23 kDa. All five subunits have the same orientation in the pentamer, with a PC binding site located on one face of each subunit. The PC binding site consists of a hydrophobic portion and two calcium ions, which are bound to CRP. Crystallographic analysis of CRP-PC complexes has demonstrated that the phosphate group of PC directly coordinates with the two calcium ions. The affinity of CRP for PC is about four orders lower than that for a typical antibody. In order to form relatively stable complexes, CRP must bind a PC conjugate through at least two binding sites (i.e. epitopes). In other words, it appears that there is a minimal-size and/or flexibility requirement for the PC carrier moiety. The PC conjugate must be able to establish simultaneous multiple interactions with the CRP molecule.

Thyroglobulin (TG) is a dimeric glycoprotein with a molecular weight of approximately 670 kD. Bovine serum alumin (BSA) has a molecular weight of approximately 67 kD. Both BSA and TG proteins can be labeled with multiple copies of PC to effectively establish simultaneous and multiple reactions with CRP, although the TG carrier protein is more effective since it is approximately 10× larger than the BSA molecule, thereby providing more surface area for binding PC sites. The BSA-PC conjugate requires higher concentrations to accomplish the same result.

The prior art has failed to appreciate the enhanced properties which can be attained when using a sandwich type assay method on a single step test strip which incorporates a detection zone containing at least one test line with various concentrations of anti-CRP antibodies and conjugate of phosphorylcholine (PC). The concentrations of antibodies and PC-conjugate are selected so as to provide each test line with a different and distinct range of sensitivity for circulating CRP.

While CRP is regularly assayed to detect or monitor a variety of inflammation-associated diseases, the prior art has failed to provide a rapid test system that utilizes minute quantities of undiluted sample for analysis of an exceedingly wide range of CRP concentrations.

### DESCRIPTION OF THE PRIOR ART

Many studies, articles and patents have been directed toward the use of various methods and compositions to detect acute-phase proteins, particularly to the detection of CRP concentrations in whole blood due to the pervasiveness of cardiovascular and other inflammatory-related disease.

WIPO Publication No. WO 03/036297 to Doyle et al. (herein incorporated by reference in it entirety), discloses a method of detecting and/or determining C-reactive protein in diverse animal species, wherein such a method comprises contacting a sample of biological fluids from a human or a non-human animal with a complex of a phosphorylated compound containing a nitrogen moiety and a label (enzyme or microsphere) which is directly detectable in a non-immunological assay, the phosphoryl moiety and the nitrogen being positioned relative to each other so as to permit binding to calcium-dependent ligand binding sites present on CRP. This publication teaches the use of both bovine serum albumin (BSA) and thyroglobulin (TG) as carrier proteins for PC, with BSA the preferred embodiment. Deegan et al., (analytical Biochemistry 312:175-181) also discloses this technology.

WIPO Publication No. WO 02/31503 and related U.S. Pre-grant Pub. No. 2002/0061600 (both herein incorporated by reference in their entirety), disclose a method and test kit for carrying out the method. The test kit comprising a test strip having a receptor-binding ligand immobilized in or on reaction zones of the membrane. These references do not disclose the use of an immunological reagent comprising anti-CRP antibodies and an non-immunological reagent comprising a PC-conjugate on a lateral flow device as disclosed in the present invention.

GB Patent Application 2217335 A, to Heggli (herein incorporated by reference in it entirety), discloses a diagnostic composition comprising phosphorylcholine residues and/or aminoethyl dihydrogen phosphate residues immobilized on a solid phase for quantitative and/or qualitative detection of CRP in a sample. PC-captured CRP can be detected with an antibody-linked detector moieties.

GB Patent Application 2217840 A, to Heggli (herein incorporated by reference), discloses a diagnostic composition comprising phosphorylcholine residues and/or aminoethyl dihydrogen phosphate chemically linked to an enzyme or a fluorescent agent or radioactive substance or metal colloid particle (preferably gold or silver) . These compositions are useful for quantitative and/or qualitative detection of CRP in a sample.

U.S. Pat. No. 6,194,225 to Oka et al. (herein incorporated by reference), teach a test strip that facilitates accurate and rapid detection of an antigen contained within a fluid sample. The test strip divides the sample into two routes, such that the antigen is captured efficiently even when the antigen is in the sample in a small amount. Moreover, the test strip is particularly effective if the antigen to be tested is CRP.

U.S. Pat. No. 6,183,972 to Kuo et al. (herein incorporated by reference), is directed to the use of a nitrocellulose test strip for determining the concentration of CRP in a body fluid. The test strip contains at least three test bands of monoclonal mouse anti-C reactive protein (CRP) antibody and one control band of polyclonal donkey anti-goat. This reference teaches the use of the detection signals generated from the amount of analyte in the sample in each reaction band, to mathematically combine these patterns to generate a monotonous dose-response curve which factors out the hook effect. This patent does not teach or suggest the incorporation of a non-immunological reagent in the capture test bands.

U.S. Pat. No. 5,003,065, to Merritt et al. (herein incorporated by reference), discloses compounds including a protonated CRP specific binding moiety (modified phosphorylcholines) complexed with a metal ion. Such compounds are compressed within a membrane. When CRP binds the electrochemical potential across the membrane undergoes a change which can be electrochemically measured. This electrochemical potential variation appears to be a direct function of the quantity of CRP bound by the compound, thus such measurement can be used to measure CRP in samples.

All of the cited prior art teaches either immunological or non-immunological reagent systems to detect the amount of analyte in a sample. What has heretofore been lacking in the art is an elegant and inexpensive diagnostic platform for detection of an analyte utilizing a combination of non-immunological and immunological reagents of greater and lesser affinity.

It has been discovered that such a mixture of non-immunological (e.g. PC-protein conjugates) and immunological (e.g. antibodies) substantially reduces the hook effect, such that if high concentrations of an analyte are present in a sample there is no corresponding decrease in the detection signal. What is particularly unique is the use of multiple capture reagents zones each comprising different concentrations of antibodies and PC-protein conjugates providing a wide detection range for use on a test strip.

### SUMMARY OF THE INVENTION

The present invention makes use of a conjugate complex for enhancing the density of phosphorylcholine (PC) sites to establish simultaneous multiple interactions with a C-Reactive Protein (CRP). The invention presented herein operates through the mechanism of binding C-Reactive Protein with a modified carrier protein. In a preferred embodiment, the carrier protein is thyroglobulin (TG) modified by the addition of multiple PC-binding sites.

Due to the properties of the TG carrier protein (i.e. large molecular weight and size), the PC-TG conjugate is able to achieve higher sensitivity in the detection of CRP as compared with other conventional carriers. Moreover, the PC-TG carrier protein can be readily incorporated onto a lateral flow platform comprising a anti-CRP antibodies, forming a immunological and non-immunological mixture, the concentrations of antibodies and PC-TG provide enhanced sensitivity for capture of CRP.

Accordingly, it is an objective of the instant invention to teach a method for detecting CRP across an entire analyte concentration range on one test strip.

It is a further objective of the instant invention to teach a composition of immunological and non-immunological reagents to capture both high and low concentrations of analyte.

It is a yet another objective of the instant invention to provide a test kit for determining the amount of analyte present in a sample mixture of reagents of the present invention immobilized on a solid phase substrate.

An additional objective of the present invention is to provide a sample testing method and composition that does not require dilution of the sample prior to testing, even when sample contains large amounts of analyte.

Other objects and advantages of this invention will become apparent from the following description, wherein are set forth, by way of illustration and example, certain embodiments of this invention.

### BRIEF DESCRIPTION OF THE DRAWINGS

FIG. 1 is a schematic representation illustrating a sandwich type assay at an Ab1 capture zone exhibiting the prozone effect using a gold conjugate detector molecule;

FIG. 2 is a schematic representation illustrating sandwich type assay at the PC-TC capture zone such that no prozone effect is observed;

FIG. 3 is a schematic representation demonstrating the reaction of immobilized second antibody with the mobile label at the control zone on the substrate.

FIG. 4 is a perspective view illustrating a representative immunochromagrapic device in accordance with the present invention.

### DETAILED DESCRIPTION OF THE INVENTION

The term “sample” as used herein refers to whole blood, serum, plasma, saliva, tear fluid, urine, cerebrospinal fluid, sweat, lymph, colostrum and other bodily fluids known to those skilled in the art.

The term “lateral flow device” generally refers to a class of devices which includes an immunochromatographic test strips capable of providing simple and rapid semi-quantitative or qualitative detection of many analytes including antigens, antibodies and products of nucleic acid amplification tests. These test strips are part of a segment of in vitro diagnostics called Point of Care (POC) devices. In general a signal reagent is solubilized and bound to an antigen or antibody in the sample, subsequent to which it moves through a membrane via capillary action. It may then bind to a specific analyte, if present, after which it further binds to a second immobilized antibody or antigen, along a test line or the like, at which point a signal forms which may be read by eye or machine.

The term “solid phase” refers to any immunochromatograpic type assay (e.g. test strip), microtiter plate, microsphere, microparticle or the like.

The term “label” refers to any one of colloidal metals (gold, silver, platinum, selenium, copper, or the like), latex particles, silica or polystyrene particles, organic dyes, pigments, metallic oxides, an lanthanide, enzymes or combinations or residues of these for detection of an analyte by the naked eye or appropriate instrumentation.

The term “PC-conjugate” used herein refers to any protein capable of being modified by the addition of multiple PC-binding sites. Non-limiting examples of proteins include thyrogobulin (TG), bovine serum albumin (BSA), casein, ovalbumin and keyhole limpet haemocyanin (KLH) and fragments, mixtures or combinations thereof.

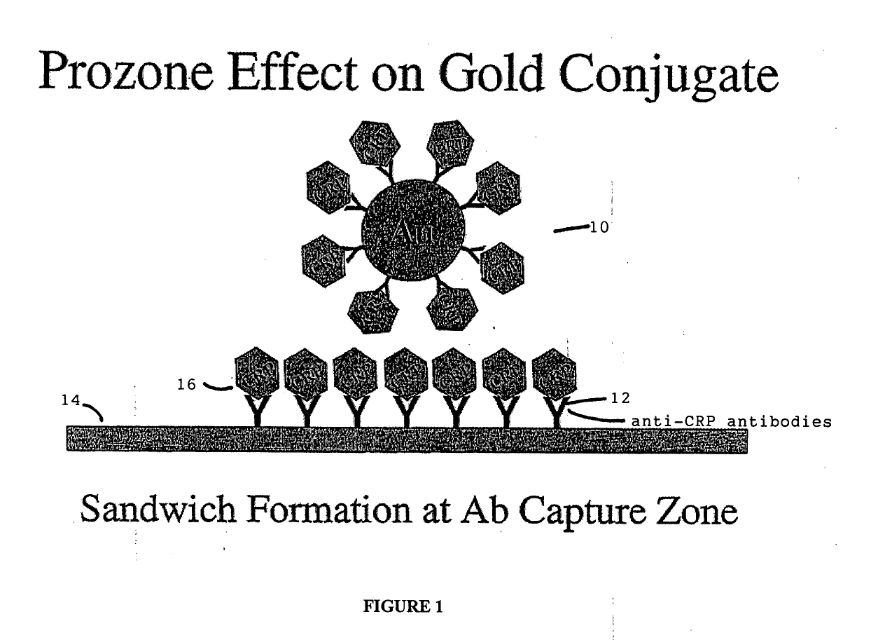

FIG. 1 illustrates the prozone effect commonly found in prior art sandwich type format. Antibodies 12 are immobilized on the solid phase substrate 14, forming the antibody complex 16 to capture the gold conjugate detector molecule 10. Both the substrate 14 and the conjugate 10 are fully saturated with the excess CRP molecules, such that the gold conjugate 10 cannot cross-link with the antibody complex 16, thus no observable reaction is formed. This can result in an underestimation of the true CRP amount and possible misdiagnosis of the individual.

Particularly preferred, for ease and simplicity of detection, and its quantitative nature, is the sandwich or double antibody assay of which a number of variations exist, all of which are contemplated by the present invention. For example, in a typical sandwich assay, unlabeled antibody is placed on a solid phase, e.g. test strip, which incorporates a matrix of bibulous (i.e. nitrocellulose or nylon membranes) or non-bibulous material (as described in U.S. Pat. No. 4,943,522, herein incorporated by reference), along which the fluid flows by capillary action. The strip can include a sample pad and/or cell separator onto which the sample is applied, such that it filters impurities present in the sample. The filtered sample flows downstream to a bind with a labeled detection reagent on the substrate. The capillary action is created by an absorbing pad located downstream of the test strip.

The labeled sample then flows to a detection zone at which at least one capturing reagent is immobilized for retention of the analyte of interest. If the sample is positive for the presence of an analyte, the labeled analyte in the detection zone produces a colored response, which is then visually inspected or detected by a reflectance or electrochemical measurement system.

The instant invention is directed towards a conjugate system and method utilizing a solid support, preferably a test strip, upon which a conjugate reagent system is immobilized thereon in a manner effective to provide an assay range from <0.1 to >100 μg CRP/mL, capable of detecting both low concentrations of CRP in a healthy individuals and high concentrations of CRP present in individuals suffering from disease. This is accomplished by providing the detection zone with least one-test line containing a mixture of polyclonal anti-bodies and PC-conjugate, particularly PC-TG conjugate. There is no limit to the number of detection zones as long as there is enough space available on the test strip. Additionally, the detection zone may include a control line to verify test completion and validity.

The synthesis of PC-TG conjugate requires the following steps:
1) attachment of thyroglobulin via sugar chains;
2) the reduction of the p-nitrophenyl phosphorylcholine to p-amino-PPC;
3) preparation of diazonium salt of APPC;
4) coupling of the above intermediate to histamine-TG form step 1;
5) dialyze or desalt the final conjugate against PBS.

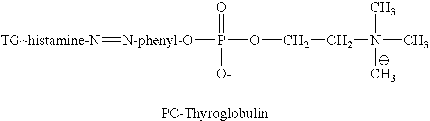

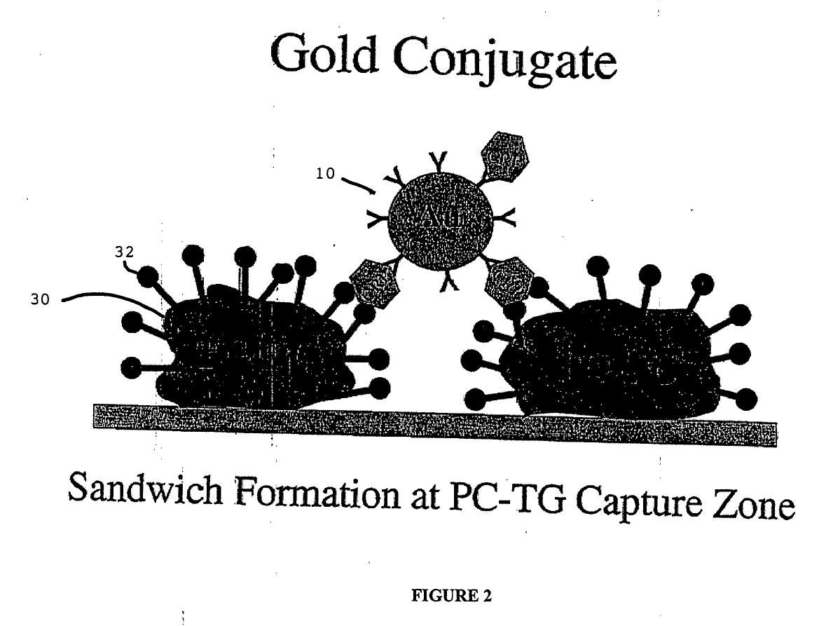

FIG. 2 illustrates a schematic representation of a sandwich type assay with phosphorylcholine thyroglobulin conjugate (PC-TG) capture zones on a substrate using a gold conjugate detection reagent attached to at least one epitope of the analyte (CRP). Due to the large size of the TG molecule 30, it can be labeled with a large density of PC binding sites 32 making it extremely effective in binding with two epitopes of the CRP analyte. Moreover, the modified TG protein of the present invention is capable of forming multiple reactions even when the gold conjugate 10 is fully saturated with CRP.

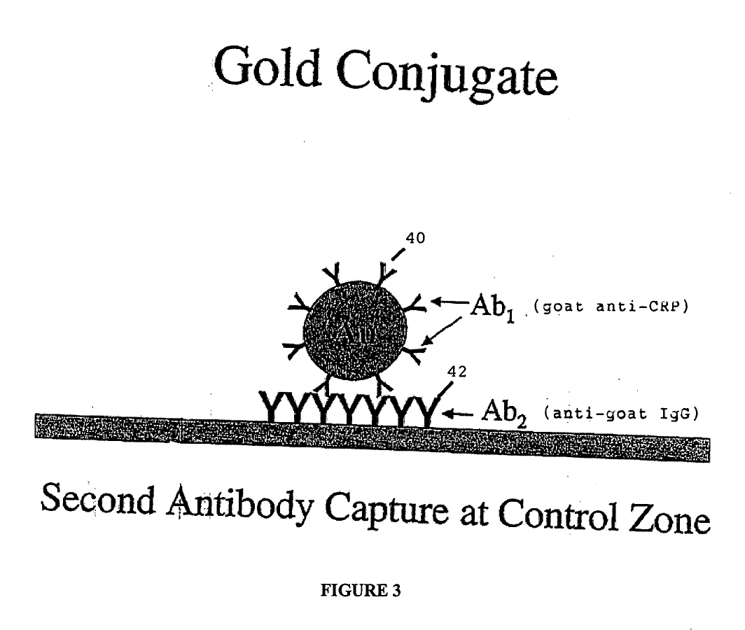

The schematic representation set forth in FIG. 3, illustrates an example of the control zone 48 which may be present on the test strip. In this case the detector reagent is a colloidal gold conjugated with affinity-purified goat anti-CRP 40, which binds to a second antibody 42 at the control zone. The second antibody 42 utilizes a donkey anti-goat IgG designated Ab2 (manufactured by Lampire Biological Laboratories). Since the gold conjugate is often in excess of the sample reactive antibodies, sufficient conjugate is available to react with the control line.

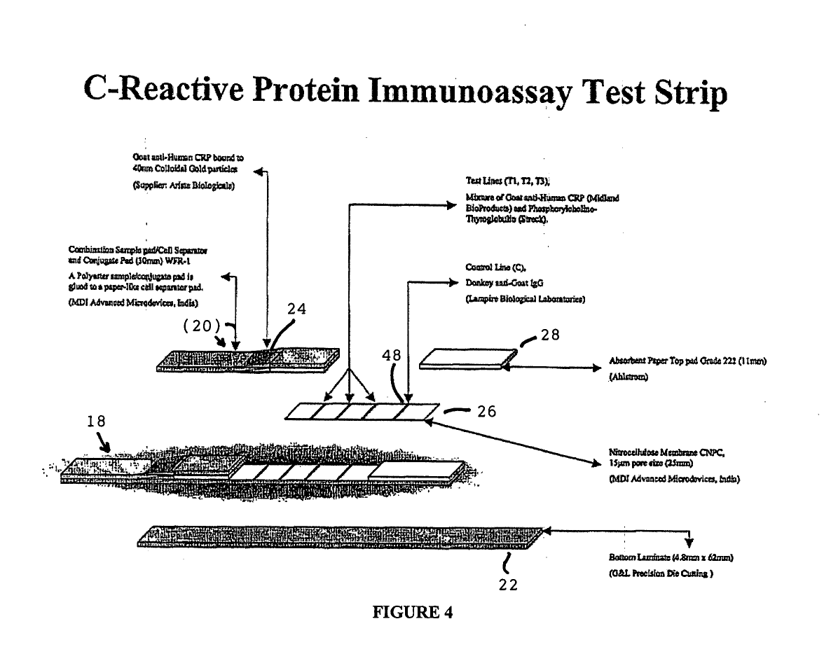

In one illustrative, albeit nonlimiting embodiment of a test strip 18 of the instant invention shown in FIG. 4, an undiluted test sample is applied to the sample pad/cell separator and conjugate pad 20 (manufactured by MDI Advance Microdevices, India) attached to a bottom laminate 22 (manufactured by G&L Precision Die Cutting). The sample wicks from the sample pad/cell separator 20 toward a detector reagent portion 24 containing an affinity-purified goat anti-CRP bound to 40 nm colloidal gold particles (supplied by Arista Biologicals). Subsequently, the bound detector reagent wicks through a nitrocellulose membrane 26 (manufactured by MDI Advanced Microdevices, India) containing three test lines T1-T3.

The three test lines, T1, T2, T3 incorporate a combination of an IgG fraction of goat anti-CRP, designated Ab1, (manufactured by Midland Bioproducts) and phosphorylcholine-thyroglobulin (PC-TG). The level of differentiation desired determines the number of test lines and amount of immobilized PC-conjugate and Ab1 reagents.

When the level of analyte in the test sample is low, generally the sandwich is formed in the first distinct test zone. The test strip is then read without any interference. However, when excessive analyte is present in the sample the Ab1 capture reagent becomes fully saturated and analyte-labeled antibody begins binding to the PC-conjugate. As the first capture zone becomes saturated, the unbound analyte-labeled antibody conjugate flows through the first capture zone and is bound to one of the subsequent reaction zones. Any excess not reacted with the test lines is absorbed by the control line containing donkey anti-goat IgG (designated Ab2).

The test lines are selected so as to provide each one with a greater and lesser binding affinity, forming a broad range of detection sensitivity for circulating CRP. An illustrative, but non-limiting example of the concentrations of the capture reagents and the detection range resulting therefrom are set forth in Table 1.

TABLE 1
DETECTION ZONE CONTAINING THREE
TEST LINES AND ONE CONTROL

| | Capture Reagents | | | |
|---|---|---|---|---|
| Line | (mg/mL) | | CRP Detection | Cardiovascular |
| Number | Antibody | PC-TG | Range (μg/mL) | Risk |
| T1 | Ab1 (0.26) | 0.5 | 0.1 to 2.0 | Low/Average |
| T2 | Ab1 (0.19) | 0.5 | 3.0 to 9.0 | High |
| T3 | Ab1 (0.16) | 1.0 | 10 to 100.0 | Active inflammation |

| | | | |
|---|---|---|---|
| C | Ab2 (2.0) | 0 | Indicates Test Completion/Validity |

The results from Table 1 were obtained using a test strip similar to that shown in FIG. 4, detected by a reflectance spectrometer (not shown). The signals produced by the label bound to each of the test lines, T1-T3 can be quantitatively detected by any detection means known in the art, i.e. reflectance, electrochemical biosensor, etc. The amount of labeled detector conjugate is then correlated to the concentration of analyte present in the sample. The results demonstrate the wide assay range for CRP detection capable in the present invention.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures. One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A composition for use on a solid-phase assay for detection of C-Reactive Protein believed to be present in a sample, the composition comprising:
an immunological reagent comprising anti-CRP antibodies; and
an non-immunological reagent comprising a conjugate of phosphorylcholine (PC);
wherein said concentrations of said anti-CRP antibodies and said PC-conjugate are selected so as to provide a range of sensitivity for detection of circulating CRP.

2. The composition of claim 1, wherein said phosphorylcholine conjugate comprises phosphorylcholine-thyroglobulin conjugate (PC-TG).

3. The composition of claim 1, wherein said phosphorylcholine conjugate comprises phosphorylcholine-bovine serum albumin conjugate (PC-BSA).

4. The composition of claim 1, wherein said phosphorylcholine conjugate comprises a mixture of phosphorylcholine-thyroglobulin conjugate (PC-TG) and phosphorylcholine-bovine serum albumin conjugate (PC-BSA).

5. The composition of claim 1, wherein said antibody comprises an IgG fraction of goat anti-CRP.

6. A method for detecting C-Reactive Protein believed to be present in a sample, comprising:
contacting said sample with a detector reagent, such that said detector reagent binds with said CRP;
saturating said composition as set forth in claim 1 with said bound detector such that said CRP is effectively captured;
detecting any captured CRP;
wherein said detected amount of captured CRP provides a quantifiable amount of CRP concentration present in the sample.

7. The method for detecting according to claim 6, wherein the sample has a concentration of about 3.0 to 100.0 μg/mL of CRP.

8. The method for detecting according to claim 6, wherein the sample is undiluted.

9. The method for detecting according to claim 6,
wherein said detector reagent comprises affinity-purified anti-CRP antibodies;
and at least one member selected from a group consisting of; a metal colloid, radioactive label, or fluorescent label, or residues thereof.

10. The method for detecting according to claim 6,
wherein said saturating step comprises contacting said bound detector with at least two distinct areas containing said composition as set forth in claim 1.

11. A diagnostic assay kit for determining the concentration of circulating CRP in a sample, comprising:
a solid-phase substrate comprising a labeled reagent for binding with said sample and having a detection zone comprising at least one area on which the composition of claim 1 is immobilized;
means for detecting said labeled amount of captured CRP on said at least one area.

12. The diagnostic assay kit of claim 11, said composition of claim 1 immobilized on three distinct areas in said detection zone, comprising:
said composition present in an amount effective in a first area to provide detection from about 0.1 to about 2.0 μg/mL of CRP;
said composition present in an amount effective in a second area capable of detecting from about 3.0 to about 9.0 μg/mL of CRP;
said composition present in an amount effective in a third area capable of detecting from about 10.0 to about 100.0 μg/mL of CRP.

13. The combination of claim 11, wherein said solid phase comprises a test strip wherein said composition of claim 1 is positioned in an amount effective to provide an assay range from about 0.1 to about 100 μg/mL CRP.

14. The diagnostic assay kit of claim 11, wherein said solid-phase substrate is at least one selected from the group consisting of a test strip, microtiter plate, microsphere, or microparticle.

15. The diagnostic assay kit of claim 11, wherein said labeled reagent comprises an IgG fraction of goat anti-CRP.

16. The diagnostic assay kit of claim 11, wherein said sample is undiluted whole blood.

17. The diagnostic assay kit of claim 11, wherein said detection zone further comprises a control of donkey anti-goat IgG.

18. The diagnostic assay kit of claim 11, wherein said means for detecting is performed by a reflectance or electrochemical measurement system.
